(19) United States
(12) Patent Application Publication
(10) Pub. No.: US 2006/0135823 A1
(43) Pub. Date: Jun. 22, 2006

(54) Process for preparing dimethylether from methanol

(75) Inventors: Ki-Won Jun, Taejon (KR); Hyun-Seog Roh, Taejon (KR); Kew-Ho Lee, Taejon (KR); Jae-Woo Kim, Choongchungnamdo (KR)

Correspondence Address: EDWARDS & ANGELL, LLP, P.O. BOX 55874, BOSTON, MA, 02205, US

(73) Assignee: SK Corporation, Seoul (KR)

(21) Appl. No.: 10/545,595

(22) PCT Filed: Apr. 10, 2003
(86) PCT No.: PCT/KR03/00720

§ 371 Date: Dec. 21, 2005

(30) Foreign Application Priority Data
Feb. 19, 2003 (KR) 10 2003 0010426

(51) Int. Cl. C07C 41/09

(52) U.S. Cl. 568/671

(57) ABSTRACT

The present invention relates to a process for preparing dimethyl ether from methanol. More particularly, this invention relates to an improved process for preparing dimethyl ether with high yield useful as a clean fuel as well as a raw material in chemical industry performed via a catalytic system, wherein dehydration of methanol is first carried out by using a hydrophilic solid acid catalyst and then subsequent dehydration of methanol is carried out continuously by using a hydrophobic zeolite solid acid catalyst in the concurrent presence of unreacted methanol, dimethyl ether produced and water.

### BACKGROUND OF THE INVENTION

1. Field of the Invention

The present invention relates to a novel process for preparing dimethyl ether, performed in such a manner that methanol is initially dehydrated over a hydrophilic solid acid catalyst and then unreacted methanol is continuously dehydrated over hydrophobic zeolite solid acid catalyst in the co-existence of the unreacted methanol and the products generated from the initial dehydration (dimethyl ether and water), which enables methanol dehydration to proceed in a more efficient manner. Therefore, the dimethyl ether useful as a clean fuel and a raw material in chemical industry may be obtained in higher yield.

2. Description of the Related Art

Dimethyl ether has been acknowledged as a principal material having diverse applicabilities in chemical industry such as aerosol propellant and it has been recently approved as a clean fuel. Further, dimethyl ether would soon be able to replace some conventional fuels used for internal combustion engines and thus development of an economic process for its preparation is in high demand in the art.

Most of the processes for preparing dimethyl ether performed in industrial scale are carried out via dehydration of methanol as represented by the following Scheme I:
2CH₃OH→CH₃OCH₃+H₂O (I)

The preparation process of dimethyl ether via dehydration of methanol is performed at a temperature of 250-450° C. and commonly uses a solid acid catalyst. The reactant is passed through a fixed reactor charged with the solid acid catalyst. The solid acid catalyst useful in the process for preparing dimethyl ether includes gamma-alumina Japanese Patent Kokai 1984-16845), silica-alumina (Japanese Patent Kokai 1984-42333) and so on. However, water is very likely to adsorb on the surface of the gamma-alumina or silica-alumina due to its hydrophilicity, which leads to lowering active site thus decreasing its catalytic activity. Therefore, where hydrophilic gamma-alumina or silica-alumina is used as a catalyst for methanol dehydration, it is generally observed that the catalyst bed at the top of reactor shows effective dehydration but that at the bottom of reactor shows lower activity due to water generated during dehydration.

In this regard, there is need in the art for developing a novel catalyst system to overcome the shortcomings of the conventional techniques and permit the preparation of dimethyl ether in higher yield. To comply with the need, the process using hydrophobic zeolite catalyst has been suggested. However, where anhydrous methanol is used as a raw material, the catalyst deactivation occurs due to coke formation (Bull. Korean Chem. Soc., 24:106(2003)).

### SUMMARY OF THE INVENTION

The present inventors have carried out intensive researches to develop a novel process to surpass, in view of the yield of dimethyl ether, the conventional processes using hydrophilic solid acid catalyst such as gamma-alumina and silica-alumina. As a result, the present inventors have discovered that a dual-charged catalyst system comprising the upper part of a reactor charged with the hydrophilic solid acid catalyst such as gamma-alumina and silica-alumina and the lower part of a reactor charged with the hydrophobic zeolite catalyst, has catalyzed methanol dehydration with greater efficiency and enabled the catalysts to exhibit high activity for a long period of time, so that dimethyl ether may be given in higher yield. That is, the present inventors have found that the dual-charged catalyst system permitting the processes, performed in such a manner that methanol is initially dehydrated over a hydrophilic solid acid catalyst and then unreacted methanol is continuously dehydrated over hydrophobic zeolite solid acid catalyst in the co-existence of the unreacted methanol and the products generated from the initial dehydration (dimethyl ether and water), has enabled methanol dehydration to proceed in a more efficient manner. Based on the novel findings described above, the present invention has been finally completed.

Accordingly, it is an object of this invention to provide a process for preparing dimethyl ether, which employs a dual-charged catalyst system comprising the upper part of a reactor charged with the hydrophilic solid acid catalyst such as gamma-alumina and silica-alumina and the lower part of a reactor charged with the hydrophobic zeolite catalyst.

### DETAILED DESCRIPTION OF THE INVENTION

In an aspect of this invention, there is provided a process for preparing dimethyl ether, which comprises the steps of: (a) dehydrating methanol by contacting with a hydrophilic solid acid catalyst; and (b) continuously dehydrating unreacted methanol by contacting with a zeolite as a hydrophobic solid acid catalyst in a state where said unreacted methanol and products generated from the step (a) coexist.

In particular, the present invention employs a dual-charged catalyst system that comprises: the upper part of a reactor charged with the hydrophilic solid acid catalyst selected from gamma-alumina and silica-alumina and the lower part of a reactor charged with the hydrophobic zeolite catalyst whose SiO₂/Al₂O₃ ratio ranges from 20 to 200. This catalyst system allows to provide more efficient methanol dehydration, thereby permitting much higher yield in dimethyl ether production.

The present invention will be described in more detail hereunder:

The present invention is directed to a novel process for preparing dimethyl ether useful as a raw material in chemical industry and a clean fuel, using the dual-charged catalyst system comprising the upper part of a reactor charged with the hydrophilic solid acid catalyst selected from gamma-alumina and silica-alumina and the lower part of a reactor charged with the hydrophobic zeolite catalyst, which enables methanol dehydration to proceed in a more efficient manner. The present process shows much higher yield of dimethyl ether. Where the dual-charged catalyst system of the present invention is used, it accompanies with higher yield of dimethyl ether and also high activity of a given catalyst can be maintained for a long period of time. Therefore, the methanol dehydration can be proceeded in a most efficient way.

The performance of the dual-charged catalyst system could be maximized when the upper part of a reactor is charged with 50-95 vol % of the hydrophilic solid acid catalyst and the lower part of a reactor is charged with 5-50 vol % of the hydrophobic zeolite catalyst.

The hydrophobic zeolite catalyst used in the lower part of a reactor includes, but not limited to, USY, Mordenite, ZSM-type zeolite, Beta and the like. According to a preferred embodiment, its SiO₂/Al₂O₃ ratio ranges from 20 to 200. If SiO₂/Al₂O₃ ratio of the zeolite is below 20, its hydrophilicity becomes manifest resulting in the catalyst deactivation due to the adsorption of water under the condition. If SiO₂/Al₂O₃ ratio of the zeolite exceeds 200, the amount of its acid site becomes negligible thus being unable to perform the efficient methanol dehydration. The hydrophilic catalyst used in the upper part of a reactor is gamma-alumina or silica-alumina.

As a result, by use of novel catalyst system for methanol dehydration, the present invention allows accomplishing higher yield of dimethyl ether than sole gamma-alumina or silica-alumina, and maintaining the higher yield for a long period of time.

In the present catalyst system described previously, gamma-alumina or silica-alumina as the hydrophilic solid acid catalyst used in the upper part of a reactor can be prepared as follows: The common catalyst available from Strem chemicals Inc. may be used as gamma-alumina. Silica-alumina catalyst may be prepared in such a manner that colloidal silica (Aldrich, 40 wt % SiO₂ solution) is impregnated into gamma-alumina catalyst (Strem chemicals) according to a conventional impregnation method and dried at 100° C., followed by calcination. Thus prepared silica-alumina comprises 1-5 wt % of silica. As the hydrophobic zeolite catalyst used in the lower part of a reactor, USY, Mordenite, ZSM-type zeolite and Beta whose SiO₂/Al₂O₃ ratio ranges from 20 to 200 may be used.

The process for preparing dimethyl ether by methanol dehydration over the dual-charged catalyst system will be generalized as follows: After the lower part of a vertical reactor, in which the fluid is to flow downward, is charged with 5-50 vol % of hydrophobic zeolite catalyst based on the total volume of the catalyst and then the upper part of the reactor is charged with 50-95 vol % of hydrophilic solid acid catalyst, the dual-charged catalyst is pretreated at 200-350° C. with flowing inert gas such as nitrogen at 20-100 ml/g-catalyst/min. The methanol is flowed into a reactor for contacting with the catalyst bed pretreated as above. At that time, the reaction temperature is maintained at 150-350° C. If the reaction temperature is lower than 150° C., the reaction rate may not be sufficient, so that the methanol conversion is decreased; however, if it exceeds 350° C., the reaction is unfavorable for production of dimethyl ether in terms of thermodynamics, so that the methanol conversion is lowered. It is preferred that the reaction pressure be maintained in the range of 1-100 atm. If the pressure is higher than 100 atm, the unfavorable conditions occur in terms of reaction operation. In addition, it is preferred that LHSV (liquid hourly space velocity) for methanol dehydration range from 0.05 to 50 h⁻¹ based on absolute methanol. If the liquid hourly space velocity is lower than 0.05 h⁻¹, the productivity may be negligible; when it exceeds 50 h⁻¹, the methanol conversion may be poor owing to shortened contact time for a catalyst.

As described previously, the present invention employs the dual-charged catalyst system comprising the layer of hydrophilic solid acid catalyst such as gamma-alumina or silica-alumina and the layer of hydrophobic zeolite in a fixed bed reactor in which the reaction fluid contacts in the order: said layer of hydrophobic zeolite, which enables methanol dehydration to proceed in a more efficient manner. Therefore, the dimethyl ether useful as a clean fuel and a raw material in chemical industry may be obtained in higher yield.

The following specific examples are intended to be illustrative of the invention and should not be construed as limiting the scope of the invention.

### EXAMPLE 1

H-ZSM-5(SiO₂/Al₂O₃=30) zeolite catalyst and gamma-alumina catalyst were separately molded to have a size of 60-80 meshes with a pelletizer. In a fixed bed reactor, in which the reaction fluid is to flow downward, the lower part was charged with 0.5 ml of the molded zeolite and the upper part was charged with 2.0 ml of the molded gamma-alumina. Then, nitrogen gas was passed into the reactor at a flow rate of 50 ml/min and the temperature of the reactor was adjusted to 270° C. The methanol was passed into the catalyst bed under a condition where a reactor temperature is 290° C., a pressure is 10 atm and LHSV is 7.0 h⁻¹. The results are shown in Table I.

### EXAMPLE 2

H-Beta zeolite catalyst and silica-alumina (silica: 1 wt %) catalyst were molded to have a size of 60-80 meshes with a pelletizer. In a fixed bed reactor, in which the reaction fluid is to flow downward, the lower part was charged with 0.25 ml of the molded zeolite and the upper part was charged with 2.25 ml of the molded silica-alumina. Then, the methanol dehydration was performed as Example 1. The results are shown in Table I.

### EXAMPLE 3

H-USY zeolite catalyst and silica-alumina (silica: 5 wt %) catalyst were separately molded to have a size of 60-80 meshes with a pelletizer. In a fixed bed reactor, in which the reaction fluid is to flow downward, the lower part was charged with 1.0 ml of the molded zeolite and the upper part was charged with 1.5 ml of the molded silica-alumina. Then, the methanol dehydration was performed as Example 1. The results are shown in Table I.

### EXAMPLE 4

H-MOR (Mordenite) zeolite catalyst and gamma-alumina catalyst were separately molded to have a size of 60-80 meshes with a pelletizer. In a fixed bed reactor, in which the reaction fluid is to flow downward, the lower part was charged with 0.5 ml of the molded zeolite and the upper part was charged with 2.0 ml of the molded silica-alumina. Then, the methanol dehydration was performed as Example 1. The results are shown in Table I.

### EXAMPLE 5

The reactions were carried out by use of the same catalyst system as Example 1 except that the temperature for methanol dehydration was changed to 250° C. The results are shown in Table I.

### EXAMPLE 6

The reactions were carried out by use of the same catalyst system as Example 1 except that the LHSV for methanol dehydration was changed to 9 h⁻¹. The results are shown in Table I.

### EXAMPLE 7

The reactions were carried out by use of the same catalyst system as Example 1 except that the temperature and LHSV for methanol dehydration was changed to 250° C. and 9 h⁻¹, respectively. The results are shown in Table I.

### COMPARATIVE EXAMPLE 1

Gamma-alumina catalyst was molded to have a size of 60-80 meshes with a pelletizer and a fixed bed reactor was charged with 2.5 ml of the molded catalyst. The methanol dehydration was carried out under the same reaction conditions as Example 1. The results are shown in Table I.

### COMPARATIVE EXAMPLE 2

Silica-alumina (silica: 5 wt %) catalyst was molded to have a size of 60-80 meshes with a pelletizer and a fixed bed reactor was charged with 2.5 ml of the molded catalyst. The methanol dehydration was carried out under the same reaction conditions as Example 1. The results are shown in Table I.

### COMPARATIVE EXAMPLE 3

H-ZSM-5(SiO₂/Al₂O₃=30) zeolite catalyst was molded to have a size of 60-80 meshes with a pelletizer and a fixed bed reactor was charged with 2.5 ml of the molded zeolite. The methanol dehydration was carried out under the same reaction conditions as Example 1. The results are shown in Table I.

### COMPARATIVE EXAMPLE 4

0.5 ml of H-ZSM-5(SiO₂/Al₂O₃=30) zeolite catalyst and 2.0 ml of gamma-alumina catalyst that were molded to have a size of 60-80 meshes with a pelletizer, were mixed and then a fixed bed reactor was charged with the mixture. The methanol dehydration was carried out under the same reaction conditions as Example 1. The results are shown in Table I.

The following Table I summarizes the results from the methanol dehydration in Examples 1-7 and Comparative Examples 1-4.

TABLE I

| | | | | | Yield of | |
|---|---|---|---|---|---|---|
| | | | | | dimethyl | |
| | | | | | ether (%) | |
| | Catalyst (vol %*) | | | | | After |
| | Lower | Upper | Temp. | LHSV | Ini- | 100 |
| Examples | part | part | (° C.) | (h⁻¹) | tial | hr |
| Ex. 1 | H-ZSM-5 | Gamma- | 290 | 7 | 90.5 | 91.1 |
| | (20%) | alumina | | | | |
| | | (80%) | | | | |
| Ex. 2 | H-Beta | 1% | 290 | 7 | 85.4 | 85.8 |
| | (10%) | silica- | | | | |
| | | alumina | | | | |
| | | (90%) | | | | |
| Ex. 3 | H-USY | 5% | 290 | 7 | 84.3 | 84.8 |
| | (40%) | silica- | | | | |
| | | alumina | | | | |
| | | (60%) | | | | |
| Ex. 4 | H-MOR | Gamma- | 290 | 7 | 88.1 | 88.6 |
| | (20%) | alumina | | | | |
| | | (80%) | | | | |
| Ex. 5 | H-ZSM-5 | Gamma- | 250 | 7 | 83.3 | 83.1 |
| | (20%) | alumina | | | | |
| | | (80%) | | | | |
| Ex. 6 | H-ZSM-5 | Gamma- | 290 | 9 | 84.4 | 84.0 |
| | (20%) | alumina | | | | |
| | | (80%) | | | | |
| H-ZSM-5 | Gamma- | | 250 | 9 | 77.2 | 77.7 |
| (20%) | alumina | | | | | |
| | (80%) | | | | | |

| | | | | | |
|---|---|---|---|---|---|
| Com. | Gamma-alumina(100%) | 290 | 7 | 67.0 | 66.8 |
| Ex. 1 | | | | | |
| Com. | 5% silica-alumina (100%) | 290 | 7 | 69.3 | 69.2 |
| Ex. 2 | | | | | |
| Com. | H-ZSM-5(100%) | 290 | 7 | 90.0 | 16.5 |
| Ex. 3 | | | | | |
| Com. | H-ZSM-5(20%) + Gamma- | 290 | 7 | 89.5 | 61.7 |
| Ex. 4 | alumina (80%) | | | | |

*representing ratio of the catalysts used in the upper and lower parts

As indicated in Table I, the methanol dehydrations using the present catalyst system in Examples 1-7 show significantly higher yields (above 80%) in dimethyl ether production and higher catalyst stability.

On the contrary, in the methanol dehydration using the gamma-alumina catalyst conventionally used in the industry and methanol as a raw material, lower yields (below 70%) in dimethyl ether production were observed (see Comparative Example 1). Where the silica-alumina was used as a catalyst, the yield in dimethyl ether production was relatively low, similar to that of gamma-alumina catalyst. Therefore, it could be understood that the present catalyst system exhibits about 10% higher yield in dimethyl ether production than sole gamma-alumina catalyst or silica-alumina.

In case of using sole H-ZSM-5 zeolite as a catalyst, although its initial activity was very high (the yield of dimethyl ether: 90%), the catalyst deactivation was manifest with time on stream due to coke formation, so that the yield of dimethyl ether was decreased to below 20% after 100 hr of reaction time (Comparative Example 3). Such operation was also observed when using the mixture of H-ZSM-5 zeolite and gamma-alumina without the localization in the bed.

Therefore, it could be appreciated that according to the present catalyst system, methanol is initially dehydrated over a hydrophilic solid acid catalyst including gamma-alumina or silica-alumina and then unreacted methanol is dehydrated by a zeolite, used as a hydrophobic solid acid catalyst, in the co-existence of the unreacted methanol and the products generated from the initial dehydration (dimethyl ether and water). During the latter dehydration, the formation of coke from the hydrophobic solid acid can be prevented by water, thus maintaining the catalyst activity.

As described above, the present invention employs the dual-charged catalyst system comprising the upper part of a reactor charged with the hydrophilic solid acid catalyst such as gamma-alumina and silica-alumina and the lower part of a reactor charged with the hydrophobic zeolite catalyst such as USY, Mordenite, ZSM-type zeolite and Beta, which enables the catalysts to exhibit high activity, thereby increasing the yield of dimethyl ether significantly.

What is claimed is:

1. A process for preparing dimethyl ether, which comprises the steps of: (a) dehydrating methanol by contacting said methanol with a hydrophilic solid acid catalyst; and (b) continuously dehydrating unreacted methanol by contacting with a zeolite, a hydrophobic solid acid catalyst, in a state where said unreacted methanol and products generated from said step (a) coexist.

2. The process according to claim 1, wherein said dehydrating is performed in a fixed bed reactor, using a double-packed catalyst bed comprising the layer of said hydrophilic solid acid catalyst and the layer of said hydrophobic zeolite catalyst, in which the reaction fluid pass into said catalyst bed to contact said hydrophilic solid acid catalyst first and then said hydrophobic zeolite catalyst.

3. The process according to claim 1 or 2, wherein said hydrophilic solid acid catalyst is gamma-alumina or silica-alumina, and said hydrophobic solid acid catalyst is a hydrophobic zeolite having the SiO₂/Al₂O₃ ratio of 20-200.

4. The process according to claim 2, wherein said dual-charged catalyst system comprises 50-95 vol % of said hydrophilic solid acid catalyst and 5-50 vol % of said hydrophobic zeolite.

5. The process according to claim 1, wherein said dehydrating is performed in a condition where a reaction temperature ranges 150-350° C., a reaction pressure ranges 1-100 atm and LHSV (liquid hourly space velocity) ranges 0.05-50 h⁻¹.
